(19) United States
(12) Patent Application Publication
(10) Pub. No.: US 2012/0240944 A1
(43) Pub. Date: Sep. 27, 2012

(54) DURABLE SKIN MARKING COMPOSITIONS

(76) Inventors: Valerie Belcher Sitterle, Smyrna, GA (US); Sue Ann Bidstrup Allen, Atlanta, GA (US); Evelina Ponizhaylo, Suwanee, GA (US)

(21) Appl. No.: 13/266,416

(22) PCT Filed: Apr. 27, 2010
(86) PCT No.: PCT/US10/32559

§ 371 Date: Jun. 11, 2012

Related U.S. Application Data
(60) Provisional application No. 61/173,067, filed on Apr. 27, 2009.

(51) Int. Cl. C08L 33/14; A61B 19/00; A01P 3/00; A01N 25/10; A01P 1/00

(52) U.S. Cl. 128/898; 514/772.4; 523/105

(57) ABSTRACT

A durable skin marking composition that remains legible and visible on the skin after being treated with an aqueous, alcohol-based solution. The skin marking composition of the present invention includes a cyanoacrylate base and a colorant. The cyanoacrylate base can be n-butyl cyanoacrylate, 2-ethyl-cyanoacrylate, 2-octyl-cyanoacrylate, or a combination of the foregoing. The colorant can be a dye, pigment, contrast agent, colored particle, fluorescent particle, radio-opaque particle, or a combination of the foregoing. The composition can further comprise one or more of viscosity modifiers/thickening agents, stabilizers, plasticizers, formaldehyde scavengers, polymerization accelerators, perfumes, adhesion promoters, and anti-bacterial, anti-fungal, anti-viral, or anti-microbial agents.

### CROSS-REFERENCE TO RELATED APPLICATIONS

This application claims the benefit of U.S. Provisional Application No. 61/173,067, filed 27 Apr. 2009, which is hereby incorporated by reference in its entirety as if fully set forth below.

### BACKGROUND OF THE INVENTION

1. Field of the Invention

The various embodiments of the present invention relate generally to skin marking compositions, and more specifically to skin marking compositions that remain visible after surgery preparation.

2. Description of Related Art

It is common to use marking pens on surgical patients, whether in human or veterinary practice, prior to surgery. Surgeons will make marks to indicate planned lines of incision, to delineate key anatomical landmarks, and to denote correct surgical sites. It is important that these marks remain visible after pre-operative preparation of the skin for surgery to insure surgical precision and to prevent wrong-site surgery.

In the past, surgeons have used ink compositions that contain harmful and deleterious toxins, such as magic markers and commercially-available felt-tip pens. A disadvantage, however, with the aforementioned marking devices and less harmful devices of the prior art is that their markings can be removed by skin preparations containing alcohol. The higher the concentration of alcohol, the more likely the markings are effectively completely removed from easy sight. Such preoperative skin preparations are already in common use. However, as these preparations have been shown to be more efficacious in reducing the number of antibiotic resistant bacteria within the surgical field when compared to non-alcohol based skin preparations, they are now being used at increasing rates.

The prevalence of alcohol-based skin preparations has lead to a recent need for a surgical marking formulation that is biocompatible, remains legible (i.e., does not smear or run), and is visible to a clinical team after a region of skin has been marked and subsequently treated with one of the preoperative preparations.

Cyanoacrylate compositions are well known and widely used as rapidly curing adhesives for various substrates, including human tissue. Particularly, cyanoacrylate compositions have been used as permanent adhesives to repair surgical lacerations to internal organs and blood vessels. Cyanoacrylate compositions have also been used to permanently seal wounds and prevent blood or other bodily fluid leakage from the wounds. Cyanoacrylate compositions have not, however, been used as a durable, yet removable, surgical marking composition. It is to such compositions that the present invention is primarily directed.

### BRIEF SUMMARY OF THE INVENTION

Briefly described, in preferred form, the present invention is a durable skin marking composition that remains legible and visible on the skin, even after being treated with a preoperative aqueous or alcohol-based solution. The present invention provides a legible composition that is removable after use. A surgeon uses the present composition to mark, for example, planned lines of incision, to delineate key anatomical landmarks, and to denote correct surgical sites. The present composition provides marks that remain visible after pre-operative preparation of the skin for surgery. The marks can then be removed by application and rubbing with a solution, preferably containing acetone or gamma-butyrolactone.

In an exemplary embodiment, the present invention is a method of marking skin for an activity comprising providing a skin marking composition comprising a cyanoacrylate base and a colorant, marking skin with the composition prior to the activity, and removing at least a portion of the composition after the activity. The activity is preferably surgery.

In another exemplary embodiment, the present invention is a method of marking skin in preparation of surgery comprising providing a skin marking composition comprising a cyanoacrylate base and a colorant, marking skin with the composition prior to surgery to provide a surgeon with at least one or planned lines of incision, to delineate key anatomical landmarks, and to denote correct surgical sites prior to the activity, wherein the marking remains visible after pre-operative preparation of the skin for surgery, and removing at least a portion of the composition after surgery. The composition is preferably removed by rubbing with a solvent selected from the group consisting of acetone and gamma-butyrolactone.

The durable composition for skin marking of the present invention preferably comprises a cyanoacrylate base and a colorant. The cyanoacrylate base can comprise n-butyl-cyanoacrylate, 2-ethyl-cyanoacrylate, 2-octyl-cyanoacrylate, or a combination of two or more the foregoing. The colorant can comprise anthraquinones, hydroxyanthraquinones, C.I., Solvent Green 5, C.I. Acid Red 50, and C.I. Acid Red 52[phthalocyaninato (2-)]copper, or a combination of two or more of the foregoing. The colorant is preferably greater than approximately 0.5% by mass of the composition.

The durable composition for skin marking can further comprise a viscosity modifier. The viscosity modifier can comprise polycyanoacrylates, acrylate resins, cellulose derivatives, poly(vinyl alkyl ethers), polycaprolactone, lactic acid, caprolactone, polyglycolic acid, lactic glycolic acid, polylactic acid, and thixotropic thickening agents. The viscosity modifier preferably comprises less than approximately 25% by weight of the durable composition for skin marking.

The durable composition for skin marking can further comprise an anionic stabilizer. The anionic stabilizer can comprise sulfur dioxide, nitric oxide, boron triflouride, sulfonic acid, lactone or a combination of two or more of the foregoing. The present composition preferably contains approximately 1-500 ppm of the anionic stabilizer.

The durable composition for skin marking can further comprise a radical stabilizer. The radical stabilizer can comprise hydroquinone, nitrohydroquinone, monomethyl ether hydroquinone, monoethyl ether hydroquinone, catechol, or a combination of two or more of the foregoing. The present composition preferably contains approximately 500-5,000 ppm of a radical stabilizer.

The durable composition for skin marking can further comprise a plasticizer. The plasticizer can comprise acetyl tri-n-butyl citrate, acetyl trihexyl citrate, dioctyl phthalate, dibutyl phthalate, dimethyl sebecate, phosphates, glyceryl triacetate, glyceryl tributyrate, dimethyl sebecate, diethyl sebacate, dioctyl adipate, dioctyl glutarate, butyl stearate, lauric acid, or a combination of two or more of the foregoing. It is preferable that the plasticizer comprises less than approximately 20% by weight of the skin marking composition.

The durable composition for skin marking can further comprise a formaldehyde scavenging compound. The formaldehyde scavenging compound can comprise alcohols, proteins, mercaptans, amines, amides, imides, sulfites, bisulfites, nitriles, cyclic ketones, or a combination of two or more of the foregoing.

The durable composition for skin marking can further comprise a polymerization accelerator. The polymerization accelerator can comprise hydroxyls, water, polyalkylene oxides, crown ethers, carboxylate, sulfur compounds, amino groups, imine groups, imide groups, amide groups, sodium phosphates, and metallo-organic compounds.

The durable composition for skin marking can further comprise an agent. The agent can comprise an anti-bacterial agent, anti-fungal agent, anti-viral agent, anti-microbial agent, or a combination of two or more of the foregoing.

The durable composition for skin marking can further comprise a perfume, and/or an adhesion promoter.

In another exemplary embodiment, the durable composition for skin marking of the present invention preferably comprises a cyanoacrylate base comprising n-butyl-cyanoacrylate, 2-ethyl-cyanoacrylate, 2-octyl-cyanoacrylate, or a combination of two or more of the foregoing, a colorant, and a viscosity modifier. The viscosity modifier of this embodiment preferably comprises polycyanoacrylates, acrylate resins, cellulose derivatives, poly(vinyl alkyl ethers), poly caprolactone, lactic acid, caprolactone, polyglycolic acid, lactic glycolic acid, polylactic acid, and thixotropic thickening agents. The durable composition for skin marking can further comprise a stabilizer, a plasticizer, a formaldehyde scavenging compound, a polymerization accelerator, an agent comprising an anti-bacterial agent, anti-fungal agent, anti-viral agent, anti-microbial agent, or a combination of two of more of the foregoing, and an adhesion promoter.

In yet another exemplary embodiment, the durable composition for skin marking of the present invention preferably comprises a cyanoacrylate base comprising n-butyl-cyanoacrylate, 2-ethyl-cyanoacrylate, 2-octyl-cyanoacrylate, or a combination of the foregoing, and a viscosity modifier, wherein the viscosity modifier is pre-imbued with a colorant.

In another exemplary embodiment, the durable composition for skin marking of the present invention preferably comprises a cyanoacrylate base and a colorant comprising dyes, pigments, inks, or a combination of the foregoing. The colorant is preferably greater than approximately 0.5% by mass of the total composition.

In another exemplary embodiment, the durable composition for skin marking of the present invention preferably comprises a cyanoacrylate base and a colorant comprising fluorescent particles, radio-opaque particles, or a combination of the foregoing. The colorant is preferably greater than approximately 0.5% by mass of the total composition.

These and other objects, features and advantages of the present invention will become more apparent upon reading the following specification.

### DETAILED DESCRIPTION OF THE INVENTION

The various embodiments of the present invention provide a durable skin marking composition that remains legible and visible on the skin after being treated with an alcohol-based solution that is dermatologically acceptable. “Dermatologically acceptable,” as used herein, generally refers to an ink that is substantially permanent or indelible while remaining non-toxic.

Although preferred embodiments of the invention are explained in detail, it is to be understood that other embodiments are contemplated. Accordingly, it is not intended that the invention is limited in its scope to the details of construction and arrangement of components set forth in the following description or illustrated in the drawings. The invention is capable of other embodiments and of being practiced or carried out in various ways. Also, in describing the preferred embodiments, specific terminology will be resorted to for the sake of clarity.

It must also be noted that, as used in the specification and the appended claims, the singular forms “a,” “an” and “the” include plural references unless the context clearly dictates otherwise. For example, reference to an ingredient is intended also to include composition of a plurality of ingredients. References to a composition containing “a” constituent is intended to include other constituents in addition to the one named.

Also, in describing the preferred embodiments, terminology will be resorted to for the sake of clarity. It is intended that each term contemplates its broadest meaning as understood by those skilled in the art and includes all technical equivalents which operate in a similar manner to accomplish a similar purpose.

Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The skin marking composition of the present invention comprises a cyanoacrylate base and a colorant. The present formulation can further comprise, for example, viscosity modifiers, plasticizers, stabilizers, accelerants, formaldehyde scavenging compounds, perfumes, adhesion promoters, anti-bacterial agents, anti-fungal agents, anti-viral agents, anti-microbial agents, or a combination of the foregoing.

The cyanoacrylate base imparts durability to the present skin marking composition, specifically for use as a marking agent for mammalian tissue, such as skin or nails. Cyanoacrylates are particularly well suited for the present invention because of their ability to rapidly polymerize at room temperature without the use of an added catalyst when applied to a substrate, and their ability to adhere well to tissue (such as mammalian skin). Once cured, cyanoacrylate is durable to exposure to water, blood, other bodily fluids, common solvents such as alcohols, and newer pre-surgical skin preparation solutions containing alcohols, iodine, and/or chlorohexadine. This provides a protective barrier in which the colorant is contained, thus limiting to fully preventing its premature removal upon exposure to such bodily fluids and/or skin preparation solutions, and renders the present formulation suitable for use as a durable marking agent for skin or other mammalian tissue.

The present invention provides a durable marking composition suitable for use on mammalian skin or tissue comprising a cyanoacrylate base. Preferred embodiments include n-butyl-cyanoacrylate, 2-octyl-cyanoacrylate, 2-ethyl-cyanoacrylate, or admixtures thereof.

Cyanoacrylate bases are widely used as rapidly curing adhesives for various substrates, including human tissue. In liquid form, cyanoacrylate exists as a monomer. When exposed to free radicals or anions, especially hydroxyl ions present in water, the monomer undergoes an exothermic hydroxylation reaction that results in rapid polymerization. Investigations using cyanoacrylates as tissue adhesives, i.e. suture replacements, have found that shorter-chain cyanoacrylate derivatives (e.g., 2-methyl-, 2-ethyl-) exhibit greater tissue toxicity than the longer chain derivatives (n-butyl-, 2-octyl-). N-butyl- and 2-octyl- compounds are the only cyanoacrylates currently approved by the U.S. Food and Drug Administration (“FDA”) for use with skin or other living human tissue in the United States, though 2-ethyl-cyanoacrylate is acceptable in the United Kingdom.

Application of cyanoacrylate to mammalian tissue, whether skin or other living tissue, has focused on development of adhesives for surgical use involving tissue adhesion or wound repair. Prior to the present invention, it was unknown to use cyanoacrylate in a skin marking composition, and suggestion of same was met with active disagreement. Typical uses conventionally include adhesives for repair of surgical lacerations to internal organs and blood vessels, as well as wound sealing to prevent blood or other bodily fluid leakage. These uses form in vivo sealants, fillers for internal cavities or voids, demonstrate slow reaction kinetics for use with mucosal type tissues, or have specific biodegradable properties. Typically, these adhesives are clear or imbued with a small amount of color from a limited class of colorants to just minimally achieve a tint. The nature of the monomeric cyanoacrylate component limits the selection of colorants and the amounts thereof that can be added. Too much colorant or the wrong class of colorant can cause premature polymerization of the composition, rendering it useless for application.

Previous work with cyanoacrylate adhesives has seen even known compatible pigments, such as anthraquinones and hydroxyanthraquinones, limited to less than approximately 1% by mass of the total composition, and typically ranging from approximately 0.05-0.2% by mass. At such low levels, the compositions of the prior art do not contain sufficient colorant to impart enough contrast against the skin to be effectively used as a distinctly visible marking agent. Preferred embodiments of the present invention, however, comprise colorant greater than approximately 0.5% by mass of the total composition. Other preferred embodiments comprise colorant greater than approximately 1% by mass of the total composition. In other preferred embodiments, the colorant is preferably greater than approximately 2.5% by mass of the total composition. The upper limit of colorant carrying capacity is dependent on the specific colorant, the cyanoacrylate monomer, the viscosity modifier, and the solvents for the viscosity modifier used in the final composition. Anionic and free radical stabilizing agents have less an affect on the colorant carrying capacity.

In the present invention, a polymeric agent can be used in formulations not otherwise sufficient to provide enough color contrast to increase the colorant-carrying capacity to a sufficient level in order to achieve contrast against human skin or other external tissue suitable for use as a surgical marking agent. A compatible polymeric component can be imbued with an increased amount of colorant, such as pigments, dyes, or other colored particles (i.e., colored in the ultraviolet, visible, or infrared, fluorescent, radio-opaque, etc.) in comparison to a monomeric cyanoacrylate. A polymeric agent can be used with a deeply colored polymeric component to increase contrast provided by the final formulation by preventing premature polymerization of the cyanoacrylate monomer that would normally be caused by such amounts of the colorant.

Additionally, depending on the polymeric component employed, the polymeric agent can also increase viscosity to provide the present formulation suitable for use as a non-bleeding marking agent. In the present invention, the polymeric agent is preferably selected to function as a viscosity modifier as well as to increase the colorant carrying capacity of the final formulation. Certain polymeric components can also act as plasticizers for the cyanoacrylate-based composition, preventing unwanted cracking of the marks produced when applied to flexible tissue, such as skin.

The colorant in the present composition can comprise, for example, dyes, pigments, contrast agents, colored particles, fluorescent materials, radio-opaque materials, or a combination of the foregoing. It should be understood that colorants can be chemically capped or otherwise coated or encapsulated to prevent premature polymerization caused by the nature of the colorant or to otherwise enhance compatibility with the polymeric base and other constituents of the present formulation. Colorants that have been approved by the FDA for use with food and food packaging also can be used. It is desirable, however, that the present colorant provides marking indications under visible light, ultraviolet light, and/or x-ray exposure. Such visibility will assist doctors who use many types of surgical equipment.

In a preferred embodiment, the pigment is selected from one or more of cyanoacrylate compatible and biocompatible compounds including, [phthalocyaninato (2-)] copper, D&C Violet No. 2 (1-hydroxy-4-[(4-methylphenyl)amino]-9,10-anthracenedione), Solvent Green No. 3 (1,4-di-p-toluidino-9,10-anthraquinone (PTA)), FD&C Yellow No. 6 (disodium 6-hydroxy-5-[(4-sulphonatophenyl)azo] naphthalene-2-sulphonate), FD&C Red No. 3 (9-(o-carboxyphenyl)-6-hydroxy-2,4,5,7-tetraiodo-3H-xanthen-3-one, disodium salt, monohydrate), FD&C Blue No. 2 (2-(13-dihydro-3-oxo-5-sulfo-2H-indol-2-ylidene)-2,3-dihydro-3-oxo-1H-indole-5-sulfonic acid disodium salt), C.I. Solvent Green 5 (3,9-Perylenedicarboxylicacid,bis(2-methylpropyl)ester), D&C Green No. 6 (1-Hydroxy-4-[(4-methylphenyl)amino]-9,10-anthracenedione), C.I. Acid Red 50 (sulforhodamine G), or C.I. Acid Red 52 (sulforhodamine B monosodium salt). Many other pigments and/or capped or otherwise encapsulated colorants can also be used to impart varying degrees of opacity, for example, fluorescence, or radio-opacity.

Additives for the cyanoacrylate base can include, among others, viscosity modifiers/thickening agents, stabilizers (anionic and/or free radical polymerization inhibitors), plasticizers, formaldehyde scavengers, polymerization accelerators, perfumes, and adhesion promoters, and can be used in exemplary embodiments of the present invention. Other adjuvants to the marking composition can include one or more anti-bacterial, anti-fungal, anti-viral, or anti-microbial agents.

Viscosity modifiers/thickening agents can be used to increase viscosity of a cyanoacrylate base, thereby controlling deposition and bleeding. Viscosity modifiers suited for cyanoacrylate include, but are not limited to polymeric agents such as poly-cyanoacrylates (polymeric alpha 2-cyanoacrylates); acrylate resins such as polyalkyl methacrylates, polyalkyl acrylates, and poly(methyl methacrylates); cellulose derivatives such as nitrocellulose, celluose acetates, and cellulose esters including cellulose acetate butyrate; poly(vinyl alkyl ethers); polycaprolactone, lactic-acid caprolactone, polyglycolic acid, lactic glycolic acid, polylactic acid; and copolymers thereof. Thixotropic thickening agents such as silica gels (e.g., fumed silica treated with silyl isocyanate) also can be used to modify the viscosity of the cyanoacrylate base. Carbon black silica can also be used as a thixotropic thickening agent, simultaneously imparting color to the formulation. Preferred embodiments for the present invention include nitrocellulose, cellulose acetate, cellulose esters, poly (methyl methacrylate), and/or polycaprolactone in amounts that do not exceed 25% by weight of the total composition.

Stabilizers, or polymerization inhibitors, are those compounds that can be added to extend the shelf-life of the present cyanoacrylate base by protecting against premature polymerization by anionic or free radical exposure. Suitable stabilizers useful for use of the present invention with human skin or other external tissues include, but are not limited to, anionic stabilizers such as sulfur dioxide, nitric oxide, boron triflouride, sulfonic acid, and lactone, and free radical stabilizers, such as hydroquinone, nitrohydroquinone, monomethyl ether hydroquinone, monoethyl ether hydroquinone, and catechol. Mixtures thereof are acceptable so long as the mixture of stabilizers does not adversely affect the desired polymerization rate and characteristics of the cyanoacrylate monomer. It is preferable that the anionic stabilizer comprises approximately 1-500 ppm of the total composition and that the radical stabilizer comprises approximately 500-5,000 ppm of the total composition Preferred biocompatible stabilizers for the present invention include sulfur dioxide and/or hydroquinone. Preferred embodiments can contain the minimal amount of stabilizing agents possible to achieve acceptable clinical shelf life as a marking agent while not adversely affecting desired polymerization properties, including cure rate.

Plasticizers can be used with cyanoacrylate compositions to impart more flexibility to the cured formulation. Suitable plasticizers include, but are not limited to, difunctional aromatic esters, phosphates, phosphonates, and mono- or difunctional alphiatic esters of acids. Specific examples include, but are not limited to, acetyl tri-n-butyl citrate, acetyl trihexyl citrate, dioctyl phthalate, dibutyl phthalate, dimethyl sebecate, phosphates such as triethyl phosphate and tri(p-cresyl)phosphate, glyceryl triacetate, glyceryl tributyrate, dimethyl sebacate, diethyl sebacate, dioctyl adipate, dioctyl glutarate, butyl stearate, lauric acid, and mixtures thereof. However, phthalates have recently come into question as potentially having biotoxic effects, especially in infants and children. Some polymeric agents can also produce a plasticizing effect when incorporated into monomeric cyanoacrylate. These include, but are not limited to, polyethylene glycol esters, polyester gluterates, and polyester adiapates. Preferred biocompatible plasticizers for the present invention are tributyl citrate, acetyl tributyl citrate, and/or butyl sterate in amounts that do not exceed 20% by weight of the total composition.

Formaldehyde is a well-known byproduct of cyanoacrylate degradation and is the primary compound leading to tissue toxicity. In the present invention, intended for external surgical marking of skin, nails, etc., this is less of a concern than for internally used cyanoacrylate adhesives. However, since a surgeon may cut though a given marking using the composition disclosed herein, thereby exposing internal living tissue to the ink, all aspects of biocompatibility should be considered. Therefore, the present invention can comprise formaldehyde scavenging agents, in free or microencapsulated form, to reduce formaldehyde concentration levels as the ink begins to degrade. Formaldehyde scavenging compounds appropriate for a cyanoacrylate base include, but are not limited to, alcohols, proteins, mercaptans, amines, amides, imides, sulfites, bisulfites, nitriles, cyclic ketones, and combinations thereof. Biocompatible formaldehyde scavenging compounds preferred for the present invention include sodium bisulfite and/or urea.

Polymerization accelerators, or initiators, can be incorporated into the present composition. For example, they can be used if the addition of other polymeric substances or solvents necessary to achieve beneficial contrast for use as a marking composition prolongs curing time beyond clinically acceptable parameters. Additionally, they can be used if other materials to enhance shelf-life or utility (i.e, stabilizers, scavengers, additional viscosity or plasticizing agents, or initiators) prolongs curing time beyond clinically acceptable parameters. Initiators for cyanoacrylate systems include, but are not limited to, hydroxyls, water, polyalkylene oxides, crown ethers, carboxylate, sulfur compounds such as thiols, molecules containing amino, imine, imide, or amide groups, sodium phosphates, and many metallo-organic compounds.

If incorporated into the cyanoacrylate base directly, a surfactant can be used to enhance dispersion of the initiator within the solution. Alternately, an initiator can also be added via the delivery system for the marking composition. In such a case, the initiator can be contained within a porous tip or coated along the internal, solution-contacting sides of applicator tip to enhance cure upon delivery to skin. An initiator can also be added extraneously as a spray or wipe to skin prior to marking with the present composition or applied as a spray after marking skin to quicken cure time.

The present composition can also be sterilized by, for example, chemical, physical, and/or irradiation methods.

Preferably, exemplary embodiments of the present invention act at room temperature, approximately 20° C., when applied to skin, nails, or other external body part. Exemplary embodiments also comprise elements that are biocompatible. Additionally, the process by which embodiments dry and polymerize via exothermic reaction is biocompatible.

In various embodiments of the present invention, suitable applicators or methods of application can be used to apply the composition that enables the user to control the deposition and thereby achieve effective use as a marking medium. The present skin marking composition can be applied using, for example, a finger, brush, sponge, spray, stylus, stencil, or marker pen.

In one example, the composition is applied using a marker pen. The marker can dispense the composition via a felt tip, fiber tip, brush tip, sponge tip, other porous tip, ball tip, gel dispensing tip, or other tips used to apply ink from reservoir barrels in marker pens. The marking pen can be scaled to a variety of shapes and sizes so long as the marking pen is capable of providing a desired mark on the tissue of a surgical patient. Because the spread of infectious disease is of concern, the marking pen can be disposable, and thus disposed of after a single use. Accordingly, one or more surgical marking pens can be packaged in a manner that is capable of providing an indication to a prospective user as to whether a marking pen has been previously used.

In another embodiment, a marker can comprise a reservoir that has an opening for delivering a suitable amount of the present composition from the reservoir to the nib. In one example, the nib is a conventional felted foam tip capable of receiving the composition stored in the reservoir and shaped and sized such that when the nib is drawn across the tissue of a surgical patient, the skin marking composition can be deposited onto the surface of the patient's tissue and leave a desired mark. In another example, the nib itself is the marking agent reservoir. In this example, the conventional felt tip nib is preloaded with a suitable amount of the skin marking composition such that the marking agent is operable only until the composition loaded into the nib is either deposited onto a desired surface or dries up after being exposed to air.

In yet another embodiment, a marker can comprise a sufficient amount of the present composition for a single use. The marker can be sized and shaped to form a friction fit within a cap member sized and shaped to cover the marking nib to prevent the composition from drying out and rendering the marking pen inoperable.

Cyanoacrylate can be removed by several solvents including acetone, methyl-ethyl-ketone, nitromethane, and gamma-butyrolactone. Of these, acetone and gamma-butyrolactone exhibit the least toxicity and sensitivity to humans. It is a characteristic of the present invention that the marks left by the present composition can be removed by application and rubbing with a solution containing acetone or gamma-butyrolactone, whether via pre-moistened and packaged wipes or direct application of the solution via a sponge applicator or cotton ball or the like.

Numerous characteristics and advantages have been set forth in the foregoing description, together with details of structure and function. While the invention has been disclosed in several forms, it will be apparent to those skilled in the art that many modifications, additions, and deletions, especially in matters of composition characteristics, can be made therein without departing from the spirit and scope of the invention and its equivalents as set forth in the following claims. Therefore, other modifications or embodiments as may be suggested by the teachings herein are particularly reserved as they fall within the breadth and scope of the claims here appended.

What is claimed is:

1. A method of marking skin for an activity comprising:
providing a skin marking composition comprising a cyanoacrylate base and a colorant;
marking skin with the composition prior to the activity; and
removing at least a portion of the composition after the activity.

2. The method of claim 1, wherein the activity is surgery.

3. A method of marking skin in preparation of surgery comprising:
providing a skin marking composition comprising a cyanoacrylate base and a colorant;
marking skin with the composition prior to surgery to provide a surgeon with at least one planned line of incision, to delineate key anatomical landmarks, and to denote correct surgical sites prior to the activity, wherein the marking remains visible after pre-operative preparation of the skin for surgery; and
removing at least a portion of the composition after surgery.

4. The method of claim 3, wherein removing at least a portion of the composition comprises application and rubbing with a solvent selected from the group consisting of acetone and gamma-butyrolactone.

5. A durable composition for skin marking, the durable composition comprising:
a cyanoacrylate base; and
a colorant.

6. The durable composition for skin marking of claim 5, wherein the cyanoacrylate base is selected from the group consisting of n-butyl-cyanoacrylate, 2-ethyl-cyanoacrylate, and 2-octyl-cyanoacrylate.

7. The durable composition for skin marking of claim 5, wherein the colorant is selected from the group consisting of anthraquinones, hydroxyanthraquinones, C.I., Solvent Green 5, C.I. Acid Red 50, and C.I. Acid Red 52[phthalocyaninato (2-)]copper.

8. The durable composition for skin marking of claim 5, wherein the colorant is greater than approximately 0.5% by mass of the durable composition for skin marking.

9. The durable composition for skin marking of claim 5, further comprising a viscosity modifier.

10. The durable composition for skin marking of claim 9, wherein the viscosity modifier is selected from the group consisting of polycyanoacrylates, acrylate resins, cellulose derivatives, poly(vinyl alkyl ethers), polycaprolactone, lactic acid, caprolactone, polyglycolic acid, lactic glycolic acid, polylactic acid, and thixotropic thickening agents.

11. The durable composition for skin marking of claim 9, wherein the viscosity modifier comprises less than approximately 25% by weight of the durable composition for skin marking.

12. The durable composition for skin marking of claim 5, further comprising an anionic stabilizer in an amount of approximately 1-500 ppm of the durable composition for skin marking, the anionic stabilizer selected from the group consisting of sulfur dioxide, nitric oxide, boron triflouride, sulfonic acid, and lactone.

13. The durable composition for skin marking of claim 5, further comprising a radical stabilizer in an amount of approximately 500-5,000 ppm of the durable composition for skin marking, the radical stabilizer selected from the group consisting of hydroquinone, nitrohydroquinone, monomethyl ether hydroquinone, monoethyl ether hydroquinone, and catechol.

14. The durable composition for skin marking of claim 5, further comprising a plasticizer selected from the group consisting of acetyl tri-n-butyl citrate, acetyl trihexyl citrate, dioctyl phthalate, dibutyl phthalate, dimethyl sebecate, phosphates, glyceryl triacetate, glyceryl tributyrate, dimethyl sebecate, diethyl sebacate, dioctyl adipate, dioctyl glutarate, butyl stearate, and lauric acid.

15. The durable composition for skin marking of claim 14, wherein the plasticizer comprises less than approximately 20% by weight of the durable composition for skin marking.

16. The durable composition for skin marking of claim 5, further comprising a formaldehyde scavenging compound selected from the group consisting of alcohols, proteins, mercaptans, amines, amides, imides, sulfites, bisulfites, nitriles, and cyclic ketones.

17. The durable composition for skin marking of claim 5, further comprising a polymerization accelerator selected from the group consisting of hydroxyls, water, polyalkylene oxides, crown ethers, carboxylate, sulfur compounds, amino groups, imine groups, imide groups, amide groups, sodium phosphates, and metallo-organic compounds.

18. The durable composition for skin marking of claim 5, further comprising an agent selected from the group consisting of an anti-bacterial agent, anti-fungal agent, anti-viral agent, and anti-microbial agent.

19. The durable composition for skin marking of claim 5, further comprising a perfume.

20. The durable composition for skin marking of claim 5, further comprising an adhesion promoter.

21. A durable composition for skin marking, comprising:
a cyanoacrylate base selected from the group consisting of n-butyl-cyanoacrylate, 2-ethyl-cyanoacrylate, and 2-octyl-cyanoacrylate;
a colorant; and
viscosity modifier.

22. The durable composition for skin marking of claim 21, the viscosity modifier selected from the group consisting of polycyanoacrylates, acrylate resins, cellulose derivatives, poly(vinyl alkyl ethers), polycaprolactone, lactic acid, caprolactone, polyglycolic acid, lactic glycolic acid, polylactic acid, and thixotropic thickening agents.

23. The durable composition for skin marking of claim 21, further comprising:
a stabilizer;
a plasticizer;
a formaldehyde scavenging compound;
a polymerization accelerator;
an agent selected from the group consisting of an anti-bacterial agent, anti-fungal agent, anti-viral agent, and anti-microbial agent; and
an adhesion promoter.

24. A durable composition for skin marking, comprising:
a cyanoacrylate base selected from the group consisting of n-butyl-cyanoacrylate, 2-ethyl-cyanoacrylate, and 2-octyl-cyanoacrylate; and
a viscosity modifier;
wherein the viscosity modifier is pre-imbued with a colorant.

25. A durable composition for skin marking, the durable composition having a colorant carrying capacity, the durable composition comprising:
a cyanoacrylate base; and
a colorant selected from the group consisting of dyes, pigments, and inks, wherein the colorant comprises greater than approximately 0.5% by mass of the durable composition for skin marking.

26. The durable composition for skin marking of claim 25, further comprising a polymeric agent to increase the colorant carrying capacity of the composition.

27. The durable composition for skin marking of claim 26, wherein the polymeric agent further acts as a viscosity modifier.

28. A durable composition for skin marking, the durable composition having a colorant carrying capacity, the durable composition comprising:
a cyanoacrylate base; and
a colorant selected from the group consisting of fluorescent particles and radio-opaque particles, wherein the colorant comprises greater than approximately 0.5% by mass of the durable composition for skin marking.

29. The durable composition for skin marking of claim 28, further comprising a polymeric agent to increase the colorant carrying capacity of the composition.

30. The durable composition for skin marking of claim 29, wherein the polymeric agent further acts as a viscosity modifier.
